(19) United States
(12) Patent Application Publication
(10) Pub. No.: US 2019/0309241 A1
(43) Pub. Date: Oct. 10, 2019

(54) PHOTOBIOREACTOR WITH DEVICE FOR EMITTING ELECTROMAGNETIC RADIATION, DEVICE FOR EMITTING ELECTROMAGNETIC RADIATION, AND METHOD FOR PROPAGATION OR CULTIVATION OF BIOLOGICAL MATERIAL, METHOD FOR PREPARING BIOLOGICAL MATERIAL AND/OR FOR PRODUCING PHARMACEUTICALS, IN PARTICULAR BIOPHARMACEUTICALS

(71) Applicant: SCHOTT AG, Mainz (DE)

(72) Inventors: Christian Ott, Ampfing (DE); Robert Hettler, Kumhausen (DE)

(73) Assignee: SCHOTT AG, Mainz (DE)

(21) Appl. No.: 16/379,161

(22) Filed: Apr. 9, 2019

(30) Foreign Application Priority Data
Apr. 9, 2018 (DE) 10 2018 108 327.0

(51) Int. Cl. C12M 1/00; C12N 1/12; C12N 13/00

(52) U.S. Cl. CPC C12M 21/02; C12M 31/10; C12N 13/00; C12N 1/12; C12M 41/06

(57) ABSTRACT

A photobioreactor with a device for emitting electromagnetic radiation, a device for emitting electromagnetic radiation for a photobioreactor, as well as methods of use, are provided. The photobioreactor includes a container for holding fluid media containing biological material and an electromagnetic radiation emitting device. The electromagnetic radiation emitting device has a housing with a source for emitting electromagnetic radiation arranged therein. The electromagnetic radiation emitting device including the housing thereof is arranged inside the container for holding biological material.

### CROSS REFERENCE TO RELATED APPLICATIONS

This application claims benefit under 35 USC X119 of German Application 10 2018 108 327.0, filed Apr. 9, 2018, the entire content of which are incorporated herein by reference.

The present application incorporates by reference the entire content of US Application No. [Attorney Reference No. 2133.390USU] filed simultaneously with the present application and the entire content of German Application 10 2018 323.8 filed Apr. 9, 2018 on which it is based.

The present application incorporates by reference the entire content of US Application No. [Attorney Reference No. 2133.391USU] filed simultaneously with the present application and the entire content of German Application 10 2018 108 325.4 filed Apr. 9, 2018 on which it is based.

### BACKGROUND

### 1. Field of the Invention

The invention relates to a photobioreactor comprising a device for emitting electromagnetic radiation and to a device for emitting electromagnetic radiation for a photobioreactor.

### 2. Description of Related Art

Methods for producing biological material, such as for example biotechnological production processes involving the cultivation of microorganisms, animal and plant cells, are of increasing importance. This applies in particular to the production of biopharmaceuticals or of functional food, designer food, or probiotics and diagnostics which can, for instance, induce fluorescent or (labeled) magnetic signals.

Microalgae, for example, are cultivated in tanks or reactors, in particular in tubular reactors, on a production scale. Depending on the product, sun light or LED light has been used conventionally. LED light is required if the light spectrum of the sun is not suitable for a sufficient product yield. Generally, the requirements on the production conditions of a product increase with the value thereof. Examples from the field of functional food are the production of fucoxanthin or astraxanthin, and also of phycoliproteins for diagnostic purposes, for example. However, this requires a specific selection of wavelengths. For astraxanthin, the growth phase or product formation phase is stimulated in the spectral range from blue to red light.

According to the prior art it is impossible to use microalgae for the production of biopharmaceuticals, since cultivation on a production scale obligatorily requires sterile production conditions.

Document DE 10 2005 012 515 B4 relates to a lighting device for bioreactors, in particular for incubators for the cultivation of phototrophic cell cultures, which is arranged outside a bioreactor. The lighting device substantially consists of a plurality of controllable light sources which emit light of different spectral ranges. This document furthermore discloses a method of variable illumination for the cultivation of phototrophic cell cultures in bioreactors, in particular in incubators, using a lighting device which comprises a plurality of light sources emitting light of different spectral ranges.

From DE 44 16 069 C2, a method and an apparatus are known for illuminating media introduced into containers for cultivating phototrophic microorganisms and for performing photochemical processes using light. The apparatus comprises side-emitting optical waveguides with a smooth surface, which are arranged on a support device for illumination purposes, and which radiate the light onto the medium to cause a conversion of the medium in the container. The illumination apparatus, the container, and the medium in the container can be thermally sterilized and re-cooled to a culturing temperature together, and the optical waveguides remain effective in a thermal operating range between −20° C. and +200° C. The light injected into the optical waveguides comes from a light source that is arranged outside the container.

Patent document DE 44 23 302 C1 discloses a device for injecting radiation energy of a light source into a photoreactor which comprises a tube of light-transmissive material, e.g. glass. For performing photochemical syntheses, a focusing holographic device is used which focuses at least one wavelength-selective portion of the radiant energy to the interior of the reactor.

German patent DE 10 2010 014 712 B3 discloses a modular tube photoreactor for photochemically treating fluid media, which comprises a central-axial irradiation unit comprising at least one radiation source. The irradiation unit is coaxially surrounded by a reactor wall made of glass and has an annular gap between the reactor wall and the irradiation unit, which provides an irradiation volume. This irradiation unit is also arranged outside the tube photoreactor, because although being surrounded by the latter it is not fluidically integrated into the fluid dynamics of the tube reactor.

### SUMMARY

The invention is based on the object to provide a photobioreactor with a device for emitting electromagnetic radiation and a device for emitting electromagnetic radiation for a photobioreactor which allow for a most efficient use of the photobioreactor and which mitigate contamination of the interior of the photobioreactor.

A photobioreactor which comprises a container for holding fluid media containing biological material, a device for emitting electromagnetic radiation having a housing with a source for emitting electromagnetic radiation arranged therein, and in which the device for emitting electromagnetic radiation including the housing thereof is disposed inside the container for holding biological material, allows not only to introduce electromagnetic radiation into the photobioreactor, but also to influence the fluid dynamics of the media held in the reactor.

A photobioreactor which comprises feedthroughs, in particular at least one feedthrough that extends through at least one wall of the photobioreactor, and in which the device for emitting electromagnetic radiation is held on at least one feedthrough, allows to route supply lines and/or control lines for the source of electromagnetic radiation from the exterior of the photobioreactor into the interior thereof and out therefrom. Particularly advantageously, such feedthroughs may be in the form of standard sparger ports, which moreover facilitates easier retrofitting of conventional photobioreactors with the device for emitting electromagnetic radiation according to the invention.

In order to meet the stringent requirements for the production of biopharmaceuticals, the material of the photobioreactor and of the device for emitting electromagnetic radiation may each be selected so as to comply with one or more of the following standards, depending on the application: FDA approved materials (ICH Q7A, CFR 211.65(a)—Code of Federal Regulations, USP Class, animal derivative free, bisphenol A free); EMA (European Medicines Agency) EU GMP Guide Part II approved materials; Sectoral chemical resistance—ASTM D 543-06; and Biocompatibility, e.g. referred to US Pharmacopeia or tests referred to ISO 10993.

It will be advantageous here, if the container for holding fluid media comprises stainless steel or is made of stainless steel. Any stainless steels may be used, especially also austenitic and ferritic stainless steels, but preferably only as far as they remain rust-free when practicing the invention.

Furthermore, titanium and Monel alloy with a high copper content may also be used, in principle, and when the material is used for the device for emitting electromagnetic radiation, in particular for the housing body thereof, it may also be enameled.

The photobioreactor may also be enameled, in particular on the inner surface of the container for holding biological material.

Preferably, the surfaces contacting the fluid containing the biological material have a roughness value of 0.8 or less.

If the devices for emitting electromagnetic radiation including the housings thereof are arranged spaced apart from a wall of the container for holding biological material, this allows to replace baffles, for example, and to particularly advantageously influence the flow of the fluid medium in terms of fluid dynamics in this way.

It will be advantageous in this case, if the photobioreactor comprises, in its interior, in particular in the interior of the container for holding biological material, at least one mounting means for fixing in particular baffles or turbulence metal sheets, and if the at least one device for emitting electromagnetic radiation is connected to at least this one mounting means.

If the photobioreactor is autoclavable together with the device for emitting electromagnetic radiation, in particular while the latter including the housing thereof is arranged inside the container for holding biological material, this permits to exclude with a very high degree of certainty a contamination with biologically active or interacting material.

For this purpose, the device for emitting electromagnetic radiation is in particular adapted to be autoclavable as well. Surprisingly, it has been found that 3,500 autoclaving cycles at 2 bar and 134° C. were possible with the device for emitting electromagnetic radiation as disclosed herein.

In the context of the present disclosure, autoclavable is understood to mean autoclavable in the sense of DIN EN ISO 14937; EN ISO 17665, which applies to medical devices.

In a particularly preferred embodiment, a plurality of devices for emitting electromagnetic radiation are arranged inside the container for holding biological material for this purpose, preferably symmetrically relative to the longitudinal axis of the container. This allows to achieve a more homogeneous illumination of the fluid medium and the biological material contained therein.

A preferred embodiment of a device for emitting electromagnetic radiation for being used in a photobioreactor comprises a housing with the source for emitting electromagnetic radiation arranged therein. In this way, the device for emitting electromagnetic radiation can provide contamination-free cultivation conditions within the photobioreactor.

The source for emitting electromagnetic radiation advantageously comprises LEDs, in particular an array of LEDs which allows to illuminate a larger volume within the photobioreactor in a defined manner. The LED arrays provide the light energy required for optimized cultivation of microalgae in the interior of the stainless steel bioreactor. Furthermore, the LED arrays are able to emit different wavelengths in a demand-oriented manner so that the process of growth and product formation phase can be selectively controlled thereby.

Also advantageously, the device for emitting electromagnetic radiation comprises at least one sensor for measuring the wavelength and/or intensity of electromagnetic radiation, and the at least one sensor for measuring electromagnetic radiation is preferably arranged in the housing, and in the operating state the sensor can be used to measure preferably the intensity and/or wavelength of incident electromagnetic radiation. The signals received from the sensor may be exploited in order to adjust the intensity of the incident electromagnetic radiation, in particular in a closed-loop controlled manner, by a feedback arrangement. The signals from the sensor can be routed through the feedthrough, in particular via control lines. The sensor can be used to measure the intensity and/or wavelength of the radiation emitted by the LEDs. As the latter undergo an aging process and/or as their emission spectrum may depend, for instance, on the ambient temperature, their emission during the operating state can be controlled with the help of the sensor. The at least one sensor may however also be used to control and/or to characterize the state of the biological material in the bioreactor. For example, through backscattering of the biological material illuminated in the operating state, the wavelength of the electromagnetic radiation received by the sensor can provide information about the biological material. If the biological material, e.g. certain algae, is illuminated with white light, for example, in particular for their growth, they will scatter back green light, for example. The intensity of the green light may depend on the concentration of the algae and/or on their growth state. This information can be used to control the processes in the bioreactor, such as nutrient supply, temperature, etc. It is of course also possible to measure fluorescence and the like.

In order to be able to correlate the radiation emitted by the radiation sources in the operating state with sensor signals, a device according to the invention also permits to supply a fraction of the radiation emitted by the radiation sources to a sensor as mentioned above during the operating state, for example using beam splitters and/or light guides. The means for directing the emitted radiation onto the sensor may be integrated in the housing.

Furthermore, the device for emitting electromagnetic radiation may also have one or more sensors for measuring the CO2 content within the bioreactor.

It is also advantageous if the bioreactor is equipped with more than one device for emitting electromagnetic radiation. This can provide improved illumination of the interior of the photobioreactor. It is in particular also possible in this case, that the sensors of the devices for emitting electromagnetic radiation are not only able to measure the backscattering from the material contained in the bioreactor, but also in transmission. In this case, a sensor of a device for emitting electromagnetic radiation would measure the radiation emitted by the other device for emitting electromagnetic radiation and influenced by the material in the bioreactor, in particular the transmitted and/or scattered radiation, so to speak.

By evaluating the illumination and/or sensor signals over time, the processes taking place in the photobioreactor can be controlled with increased accuracy and in particular in response to the results of the reactions of the biological material contained therein. This can contribute to a considerable improvement in yield. Generally, the actual intensity in the fluid can advantageously be controlled even in a spatially resolved manner, in particular even with a dynamic adaptation to the target intensity. Furthermore, the varying illumination depth which is caused by the changing/increasing cell density during cultivation can be compensated for. Compensation is also possible for substrate consumption and product formation which may cause a conditional change of adsorption in the fluid.

If the LEDs and/or the at least one sensor are arranged behind one or more windows of the housing, the housing of the device for emitting electromagnetic radiation can be made mechanically stable and thermally resistant, so that autoclaving as well as cleaning and sterilization can be performed using procedures that are known as CIP (Clean-In-Place) and SIP (Sterilization-In-Place).

For fluid-tightness and in particular hermeticity of the device for emitting electromagnetic radiation it is of great advantage if one or more windows have a glass seal, preferably a Glass-to-Metal Seal (GTMS) type compression glass seal. This considerably enhances mechanical stability, chemical resistance and thermal resistance.

In a preferred embodiment, at least one transparent element of a window has a sheet-like shape and has in particular plane-parallel main surfaces.

If, however, the transparent element is one of plano-convex, plano-concave, biconvex, biconcave, convexo-concave, or concavo-convex, it is possible to selectively influence the spatial distribution of the radiation field of the electromagnetic radiation and to achieve an even more homogeneous illumination of the container of the photobioreactor.

The transparent element of the respective window preferably comprises glass or is made of glass. In this case, the glass of the transparent element of the window may comprise or consist of quartz glass, borosilicate glass or glass systems which exhibit high chemical resistance, primarily to water, with good optical transmittance in the desired and subsequently stated wavelength range.

The transparent element of at least one window may exhibit a transmittance of greater than 80%, most preferably greater than 90%, in a spectral range between 250 and 2000 nm.

Economically efficient and precise manufacturing will be facilitated if the housing comprises a housing body which is formed as a milled part.

Preferably, a main body of the window is directly connected to the housing body in a hermetically sealed manner, in particular by laser welding, and the housing is hermetically sealed. In this way, the requirements on material obligatory for the pharmaceutical sector can advantageously be met.

For a largest possible illuminated volume of the container, the housing body may have a columnar shape, advantageously with a triangular cross-sectional shape, and two of the longitudinally extending side walls thereof may enclose an angle of 30°, and two of the longitudinally extending side walls thereof may enclose an angle of 60°. For the best possible illumination, a bioreactor advantageously comprises four devices for emitting electromagnetic radiation with such housing bodies.

In this case, at least two of the longitudinally extending side walls may have windows.

For a modular design, the housing of the device for emitting electromagnetic radiation may comprise at least one electrical connector which is configured to be preferably serially connected to a connector of a further housing. In order to ensure autoclavibility of the modules, the connectors are also made with gas-tight glass or ceramic insulators. The modular design with connectors allows to serially connect the arrays for a flexible adaptation to industry standard bioreactor dimensions and thus also supports upscaling.

In this case, the housing may be configured for being coupled serially in a modular way and may in particular comprise fastening means for modular serial coupling.

It will be advantageous if a pressure prevailing within the housing of the device for emitting electromagnetic radiation is reduced relative to the pressure in the exterior of the housing, and if the housing in particular has an evacuation port that can be sealed fluid-tightly, because in this case the housing can be evacuated using the evacuation port so as to have a reduced pressure compared to its exterior, so that heat conduction from the housing of the device for emitting electromagnetic radiation to assemblies arranged therein can be reduced.

As a result, electronic assemblies are in particular able to withstand, without failure, elongated time periods during which the housing is exposed to elevated temperatures, for example during sterilization or autoclaving.

Surprisingly, it has been found that the device for emitting electromagnetic radiation disclosed herein survived a number of more than 3,500 autoclaving cycles without failure.

In the context of the present disclosure, autoclavable is understood to mean autoclavable in the sense of DIN EN ISO 14937; EN ISO 17665, which applies to medical devices.

A preferred method for propagation or cultivation of biological material comprises the introducing of biological material or of a precursor of biological material into a photobioreactor as described herein, and the exposing to electromagnetic radiation emitted by a device for emitting electromagnetic radiation as described herein.

Advantageously, a method for preparing biological material and/or for producing pharmaceuticals, in particular biopharmaceuticals, comprises providing a photobioreactor which is equipped with at least one device for emitting electromagnetic radiation as disclosed herein.

If the photobioreactor has at least one feedthrough, in particular at least one standard sparger port, and if supply lines and/or control lines for the device for emitting electromagnetic radiation are routed through the at least one feedthrough, it will frequently even be possible to retrofit conventional photobioreactors or else conventional bioreactors with the presently disclosed device for emitting electromagnetic radiation in a simple and cost-effective manner.

It is particularly advantageous in this case if the photobioreactor has at least one mounting means for fixing baffles or turbulence metal sheets in its interior, in particular in the interior of the container for holding biological material, and if the at least one device for emitting electromagnetic radiation is connected to at least this one mounting means, wherein the device for emitting electromagnetic radiation is preferably fixed in the interior of the photobioreactor, more particularly in the interior of the container for holding biological material through the at least one feedthrough and the at least one mounting means.

For single-use applications, the device for emitting electromagnetic radiation may have a magnetic and/or adhesive fixation with inductive energy coupling and signal transmission.

A particularly favorable advantage for the user arises when the photobioreactor is sterilized after having been equipped with the device for emitting electromagnetic radiation. This permits to largely avoid any contamination of the photobioreactor after its sterilization.

It is furthermore advantageous if the device for emitting electromagnetic radiation has at least one sensor and the sensor can be used during the operating state to preferably measure and/or adjust the intensity and/or wavelength of incident electromagnetic radiation, in particular in a closed-loop controlled manner, and if the radiation intensity and/or wavelength of the electromagnetic radiation inside the bioreactor is measured with this sensor during the operating state.

If electromagnetic radiation of a defined wavelength, preferably 250 nm, is irradiated into the photobioreactor for a defined period of time, and a radiation intensity and/or a wavelength of the electromagnetic radiation is measured inside the bioreactor over a broad range of wavelengths or selectively at a particular wavelength, in particular at 270 nm, this allows to detect fractions of the light emitted due to fluorescence and to evaluate them with regard to specific metabolic processes within the bioreactor.

Furthermore, particularly advantageously, electromagnetic radiation of a predefined wavelength, preferably 250 nm, can be irradiated into the photobioreactor for a predefined period of time, for example during a production phase, and after this irradiation, electromagnetic radiation of a predefined further wavelength, preferably in a range from 620 to 780 nm, can be irradiated into the photobioreactor.

It is advantageous if according to one embodiment of the method the photobioreactor with its container for holding fluid media containing biological material and with the device for emitting electromagnetic radiation is autoclaved while the device for emitting electromagnetic radiation with its housing is arranged inside the container for holding biological material, because in this case contamination of the photobioreactor can be avoided with much higher probability than hitherto, and the autoclaving can be performed within a single operation. Consequently, if the autoclaving is carried out shortly before the photobioreactor is filled with the biological material, the risk of an interim introduction of contaminants is also reduced.

With the methods presently disclosed it is possible to use in particular photo- or mixotrophic microorganisms modified by mutagenesis, in particular also microalgae, yeasts, and bacteria.

The article “A novel histone crosstalk pathway important for the regulation of UV-induced DNA damage repair in Saccharomyces cerevisiae” by Boudoures, A. L., Pfeil, J. J., Steenkiste, E. M., Hoffman, R. A., Bailey, E. A., Wilkes, S. E., Higdon, S. K., Thompson, J. S., (2017) Genetics, 206 (3), pp. 1389-1402 reports about cellular repair mechanisms which can provide increased yields in the propagation of biological material.

A preferred general mechanism may be the destabilization of amines at certain irradiated wavelengths here, which leads to increased yields of the respective affected cell, through appropriate repair mechanisms.

These mechanisms can be adequately addressed by the present device, for example by irradiating electromagnetic radiation of a wavelength of 250 nm into the container for holding biological material, as will be described in more detail below.

Another example of a possibility to influence signal cascades is described in “Inhibitory effects of ginsenosides on basic fibroblast growth factor-induced melanocyte proliferation”, Lee, J. E., Park, J. I., Myung, C. H., Hwang, J. S., (2017) Journal of Ginseng Research, 41(3), pp. 268-276.

On the one hand it is stated that proliferation is stimulated by UV-B, which can also be accomplished based on the presently described methods.

On the other hand, an inhibition may be caused by adding antibodies (potentially through a feed channel of the bioreactor), and the activation by selected wavelengths of the LED array as well as the inhibition by the respective feed can be detected using a sensor unit, in particular the sensors described herein.

“ROS and calcium signaling mediated pathways involved in stress responses of the marine microalgae Dunaliella salina to enhanced UV-B radiation”, Zhang, X., Tang, X., Wang, M., Zhang, W., Zhou, B., Wang, Y. (2017) Journal of Photochemistry and Photobiology B: Biology, 173, pp. 360-367, describes that lysis of microalgae increases with increasing UV-B intensity. This reduction in cell density is accompanied by an increase in certain proteins, which allows to selectively control and increase the product formation of these proteins using the devices and methods described herein.

“Model-supported phototrophic growth studies with Scenedesmus obtusiusculus in a flat-plate photobioreactor”, Koller, A. P., Leo, H., Schmid, V., Mundt, S., Weuster-Botz, D., (2017) Biotechnology and Bioengineering, 114(2), pp. 308-320 discusses parameter-dependent growth kinetics. The parameters discussed are luminous flux intensity, cell-line-dependent adsorption and scattering, and indirectly the cultivation time. Increased biomass yield through optimization of radiation intensity is reported. This confirms the benefits of a control loop of LED arrays and a sensor unit as disclosed herein.

The described embodiments in particular also allow for mixotrophic cultivation, and photosynthesis and chemosynthesis can advantageously be combined. This also provides for a sterile and improved production of pharmaceuticals with microalgae.

The article “Adhesion of Chlamydomonas microalgae to surfaces is switchable by light”, by Christian Titus Kreis, Marine Le Blay, Christine Linne, Marcin Michal Makowski, and Oliver Baumchen, NATURE PHYSICS DOI: 10.1038/NPHYS4258 describes the behavior of microalgae which could be caused to adhere to or detach from surfaces in response to irradiation of light. By selective irradiation of electromagnetic radiation of appropriate wavelength it is now possible to achieve, for example in photobioreactors which are used in repeated batch operation, that during the change of a medium, such as nutrient solution, these microalgae adhere to surfaces and re-detach from the surface after the medium has been supplied. This also allows to further increase the effectiveness of the operation of photobioreactors.

In their publication (Phytochromes and gene expression), “Binding of phytochrome B to its nuclear signaling partner PIF3 is reversibly induced by light”, Nature 400, 781-784, the authors Ni, M., Tepperman, J. M., and Quail, P. H. (1999) describe that gene expression can be specifically induced by light, via the respective cell signaling pathways.

The publication “A red/far-red light-responsive bi-stable toggle switch to control gene expression in mammalian cells”, by Konrad Müller, Raphael Engesser, Stephanie Metzger, Simon Schulz, Michael M. Kämpf, Moritz Busacker, Thorsten Steinberg, Pascal Tomakidi, Martin Ehrbar, Ferenc Nagy, Jens Timmer, Matias D. Zubriggen, and Wilfried Weber, Nucleic Acids Research, 2013, Vol. 41(7):e77, describes this mechanism for mammalian cells for controlling transgenic activity.

Optogenetic control is also described in “A Phytochrome-Derived Photoswitch for Intracellular Transport”, by Max Adrian, Wilco Nijenhuis, Rein I. Hoogstraaten, Jelmer Willems, and Lukas C. Kapitein, DOI: 10.1021/acssynbio.6b00333, ACS Synth. Biol. 2017 Jul. 21; 6(7):1248-1256. According to this document, intracellular bi-directional transport mechanisms can be controlled in a wavelength-specific manner.

### DESCRIPTION OF THE DRAWINGS

The invention will be described in more detail below by way of preferred embodiments and with reference to the accompanying drawings, wherein:

FIG. 1 is a schematic longitudinal section through the center of a photobioreactor according to a first preferred embodiment in a vertical direction along sectional plane A-A indicated in FIG. 8, and in which an upper portion delimiting the container for holding biological material to the upper side is shown greatly simplified, for the sake of clarity;

FIG. 2 is a schematic side elevational view of a photobioreactor of the preferred first embodiment, in which an upper portion delimiting the container for holding biological material to the upper side is shown greatly simplified, for the sake of clarity;

FIG. 3 is a schematic cross-sectional view taken horizontally through the photobioreactor of the first preferred embodiment along sectional plane B-B indicated in FIG. 2;

FIG. 4 is a cross-sectional view taken horizontally through the photobioreactor of the first preferred embodiment along sectional plane C-C indicated in FIG. 2;

FIG. 5 is a more detailed and enlarged view of a section of FIG. 4, but shown slightly rotated relative to FIG. 4, illustrating the sparger port and a device for emitting electromagnetic radiation held thereon by a feedthrough and holding device;

FIG. 6 is a view of an obliquely arranged photobioreactor of the first preferred embodiment obliquely from above, in which the upper portion delimiting the container for holding biological material to the upper side has been omitted, for the sake of clarity, and the container for holding biological material is shown transparently;

FIG. 7 is a schematic perspective view, obliquely from above, of a portion of a photobioreactor of the first preferred embodiment arranged upright, in which an upper portion delimiting the container for holding biological material to the upper side has been omitted, for the sake of clarity, and the container for holding biological material is shown transparently;

FIG. 8 is again an enlarged view of FIG. 4, but shown slightly rotated relative to FIG. 4, for describing the emitted electromagnetic radiation and measured electromagnetic radiation;

FIG. 9 is a schematic sectional view taken horizontally through the device for emitting electromagnetic radiation along a sectional plane corresponding to the sectional plane of FIG. 3 and showing a detail of the photobioreactor obliquely from above, while the container for holding biological material is shown transparently;

FIG. 10 is a schematic longitudinal section through the center of a photobioreactor according to the first preferred embodiment in a vertical direction along the sectional plane D-D indicated in FIG. 8 and in which an upper portion delimiting the container for holding biological material to the upper side has been omitted, for the sake of clarity, and the container for holding biological material is shown transparently;

FIG. 11 is a detail of the sectional view of FIG. 10 as indicated by reference letter E in FIG. 10;

FIG. 12 is a detail of the sectional view of FIG. 10 as indicated by reference letter F in FIG. 10;

FIG. 13 shows an embodiment of a source for emitting electromagnetic radiation comprising a two-row array of LEDs and a two-row array of sensors as well as two controllers for driving the LEDs and for communicating with the sensors;

FIG. 14 shows an embodiment of a source for emitting electromagnetic radiation which comprises a one-row array of LEDs and a one-row array of sensors as well as two controllers for driving the LEDs and for communicating with the sensors;

FIG. 15 is a schematic sectional view along an axis of symmetry S of a window described herein for explaining the method of producing such a window;

FIG. 16 is a schematic sectional view taken along the axis of symmetry S of a window of the device for emitting electromagnetic radiation, showing part of the housing body of the device for emitting electromagnetic radiation and a plane-parallel transparent element;

FIG. 17 is a schematic sectional view taken along the axis of symmetry S of a window of the device for emitting electromagnetic radiation, showing part of the housing body of the device for emitting electromagnetic radiation and a biconvex transparent element;

FIG. 18 is a schematic sectional view taken along the axis of symmetry S of a window of the device for emitting electromagnetic radiation, showing part of the housing body of the device for emitting electromagnetic radiation and a biconcave transparent element;

FIG. 19 is a schematic sectional view taken along the axis of symmetry S of a window of the device for emitting electromagnetic radiation, showing part of the housing body of the device for emitting electromagnetic radiation and a plano-convex transparent element;

FIG. 20 is a schematic sectional view taken along the axis of symmetry S of a window of the device for emitting electromagnetic radiation, showing part of the housing body of the device for emitting electromagnetic radiation and a plano-concave transparent element;

FIG. 21 is a schematic sectional view taken along the axis of symmetry S of a window of the device for emitting electromagnetic radiation, showing part of the housing body of the device for emitting electromagnetic radiation and a convexo-concave transparent element;

FIG. 22 is a schematic sectional view taken along the axis of symmetry S of a window of the device for emitting electromagnetic radiation, showing part of the housing body of the device for emitting electromagnetic radiation and a concavo-convex transparent element;

FIG. 23 is a schematic sectional view along an axis of symmetry S of a window described herein, in which the transparent element comprises quartz glass, for explaining the method of producing such a window;

FIG. 24 is a top plan view of the window shown in FIG. 23, in which the transparent element comprises quartz glass; and

FIG. 25 is a cross-sectional view through part of the housing of the upper or first module of the device for emitting electromagnetic radiation or else of the housing of the lower or second module of the device for emitting electromagnetic radiation.

### DETAILED DESCRIPTION

The invention will be described in more detail below with reference to preferred embodiments.

In the figures which are not drawn to scale, for the sake of clarity, the same reference numerals designate the same or equivalent components.

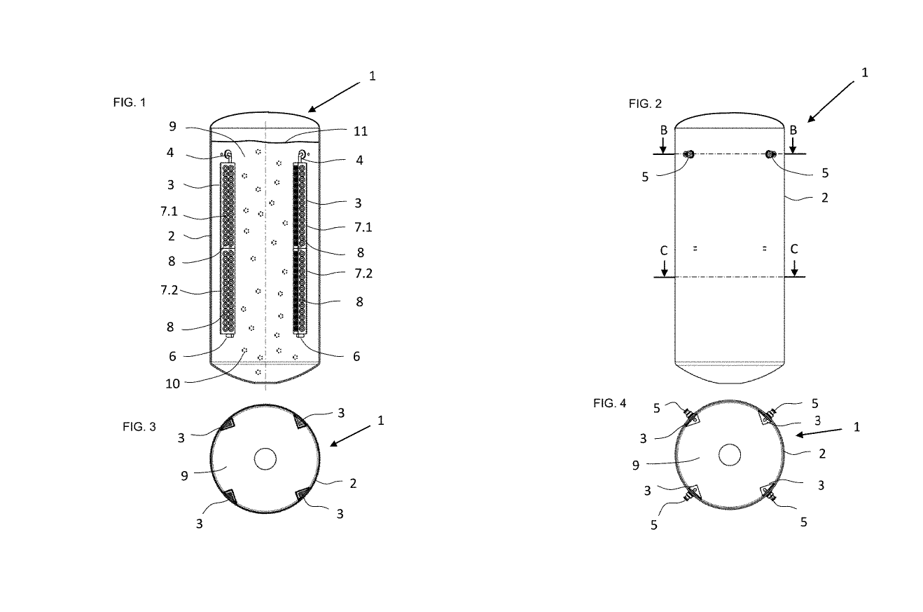

Reference is now made to FIGS. 1 and 2, each of which shows an upright photobioreactor 1 according to a preferred first embodiment in its preferred operating position.

Any positional information below refers to this orientation of the photobioreactor, and terms such as upper, lower, lateral, above or below are based on this orientation of the photobioreactor in the context of the present disclosure.

The photobioreactor 1 comprises a container 2 for holding fluid media containing biological material, which is also referred to as a container 2 for holding biological material below, or even only as a container 2.

The container 2 of the preferred embodiments is made of stainless steel or comprises stainless steel. The stainless steel may comprise or may entirely be made of 316L pharmaceutical grade steel.

Inside the container for holding biological material 2, at least one device 3 for emitting electromagnetic radiation is arranged, which comprises a housing 7 that is held at a sparger port, preferably a standard sparger port 5 at its upper end, by a feedthrough and holding device 4.

Sparger ports 5 which are known to those skilled in the art define feedthroughs 5a which extend through at least one wall of the photobioreactor, in particular through the wall of container 2, as will be described in more detail below with reference to FIG. 11. In this way, the device for emitting electromagnetic radiation 3 is held on at least one feedthrough of the photobioreactor.

At the lower end of the device 3 for emitting electromagnetic radiation, it is held on the inner wall of the container 2, by at least one mounting means 6. The mounting means 6 may be a mounting means as commonly used for fixing in particular baffles or turbulence metal sheets and is conventionally used in bioreactors or photobioreactors, such as an Inforce HT adapter, or may comprise an own standardized seat for mounting, for example a holder provided with a through-hole and held on a bolt protruding from the container 2 of the photobioreactor. By way of example only, a respective mounting means 6 with its through-hole is indicated in FIG. 1.

The device 3 for emitting electromagnetic radiation furthermore comprises a housing 7, and in the preferred first embodiment a housing 7.1 of an upper module and a housing 7.2 of a lower module, each one having at least one window 8 for transmitting electromagnetic radiation into the housing 7.1, 7.2 or out of the housing 7.1, 7.2.

By way of example, only one window 8 is indicated by a reference numeral in FIG. 1, however, it can be seen that each of the housings 7.1 and 7.2 has a plurality of windows 8.

Instead of two modules, each one with a respective housing 7.1, 7.2, the device 3 for emitting electromagnetic radiation of a further preferred embodiment comprises only one housing which has the length and number of windows of the two modules shown in FIG. 1, or which may alternatively have smaller dimensions in the longitudinal direction and a smaller number of windows. In a further embodiment of the invention, the device 3 may comprise more than two modules.

As can be seen particularly well in FIGS. 9, 11, and 12, the device 3 for emitting electromagnetic radiation is arranged with its housing 7, 7.1, 7.2 spaced apart from the wall, in particular the inner wall of the container 2 for holding biological material. In each case, a minimum distance 12 is observed between the housing 7, 7.1, 7.2 and the wall of the container 2 indicated by a double arrow in each of these figures, which ensures that fluid medium 9 containing biological material 10 can flow around the housings 7, 7.1, and 7.2 of the device 3 on all sides, so that the device 3 can take over the function of a baffle or turbulence metal sheet.

It can also be seen that in its preferred position the device 3 is completely immersed in the fluid medium 9 containing biological material 10, since the surface 11 of the fluid medium 9 is preferably at least above the windows 8.

The biological material, which is designated by reference numeral 10 only once in FIG. 1, by way of example, and is shown in a large magnification for the sake of recognizability, generally comprises photo-, hetero-, and mixotrophic microorganisms, prokaryotic and eukaryotic cells, in which photons trigger cell signal pathways, and is for example in the form of microalgae such as blue-green algae, or bacteria such as cyanobacteria, for example, and in particular also comprises photo- or mixotrophic microorganisms modified by mutagenesis, and also yeasts.

The fluid material may be in the form of an aqueous solution and may include nutrients for the biological material, such as sugars.

Referring now to the sectional view of FIG. 3, which shows that a plurality of devices 3 for emitting electromagnetic radiation are arranged within the container 2 for holding biological material, in particular four in the first preferred embodiment, which are arranged symmetrically to the longitudinal axis of the container 2.

The cross-sectional view of FIG. 4 shows the respective position of the sparger ports 5 each defining a feedthrough 5a through the wall of the container 2 of the photobioreactor 1. The respective device 3 for emitting electromagnetic radiation is held on the wall of the container 2 by a feedthrough and holding device 4 which will be described in more detail below with reference to FIG. 11, and therefore it may also be retrofitted in conventional photobioreactors which have such sparger ports.

As can be seen from the sectional view of FIG. 11, the substantially L-shaped and hollow, preferably tubular feedthrough and holding device 4 extends through the feedthroughs 5a defined by the sparger ports 5 and may have a connector 21 on its end facing the exterior of the photobioreactor.

The feedthrough and holding device 4 is secured in the sparger ports 5 and thus in the feedthroughs 5a defined thereby in a sealed manner using appropriate sealing means known to those skilled in the art, for example O-rings.

So, retrofitting and an exchange of the device 3 for emitting electromagnetic radiation is made possible by introducing into or removing from the sparger port 5 the feedthrough and holding device 4 with the device 3 attached thereto.

For a permanent attachment of the device 3 for emitting electromagnetic radiation, the feedthrough and holding device 4 may as well be connected to the sparger port 5b in a permanently and hermetically sealed manner, for example by soldering using a gold solder.

Reference is now made to FIG. 5 which shows a more detailed and enlarged view of a section of FIG. 4, but slightly rotated compared to FIG. 4.

It can be seen that the device 3 for emitting electromagnetic radiation shown in a top plan view is held on the sparger port 5 by a feedthrough and holding device 4.

Furthermore, the triangular cross-sectional shape of the columnar device 3 is apparent from this view.

The respective housing 7, 7.1, 7.2 with its columnar housing body 7.3 defines three side walls 7.4, 7.5, and 7.6. extending in the longitudinal direction thereof. Preferably, the respective housing body 7.3 may be formed of one or more milled parts.

The housing body 7.3 is preferably made of stainless steel or at least comprises stainless steel.

Any stainless steels may be used here, especially also austenitic and ferritic stainless steels, but preferably only as far as they remain rust-free when practicing the invention.

The stainless steel may preferably also comprise or entirely consist of 316L pharmaceutical grade steel.

Furthermore, titanium and Monel alloy with a high copper content may also be used, in principle, and when the material is used for the device for emitting electromagnetic radiation, in particular for the housing body thereof, it may also be enameled.

Alternatively, the housing body 7.3 may also comprise or be made of a high temperature resistant plastic, in particular a thermoplastic material such as polyaryletherketone, in particular polyetheretherketone, PEEK.

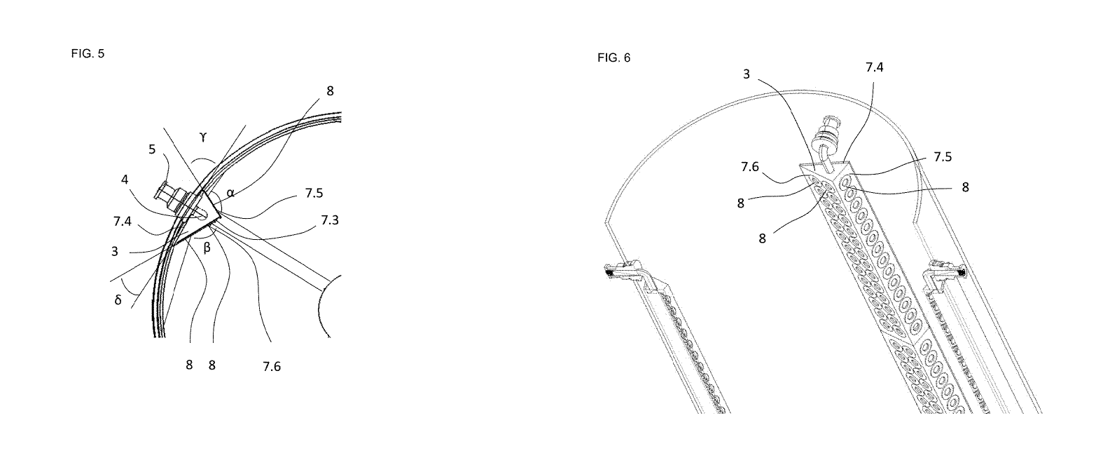

At least two of the longitudinally extending side walls 7.4, 7.5, and 7.6 each comprise windows 8, as can be seen particularly well in the view of FIGS. 6 and 7. These two side walls are the side walls 7.5 and 7.6, by way of example, as shown in each of FIGS. 6 and 7.

Two of the longitudinally extending side walls 7.4, 7.5, and 7.6 enclose an angle of 30°, and two of the longitudinally extending side walls 7.4, 7.5, and 7.6 enclose an angle of 60°.

Depending on a respectively employed agitator member and on the number of devices for emitting electromagnetic radiation described herein, other angles than 30° angles and 60° angles may be used advantageously as well.

The angles given above may also be specifically adapted to existing agitator members and then have other amounts.

In the embodiment shown in FIG. 5, side walls 7.4 and 7.5 enclose an angle γ which is 60°, and side walls 7.4 and 7.6 enclose an angle δ which is 30°.

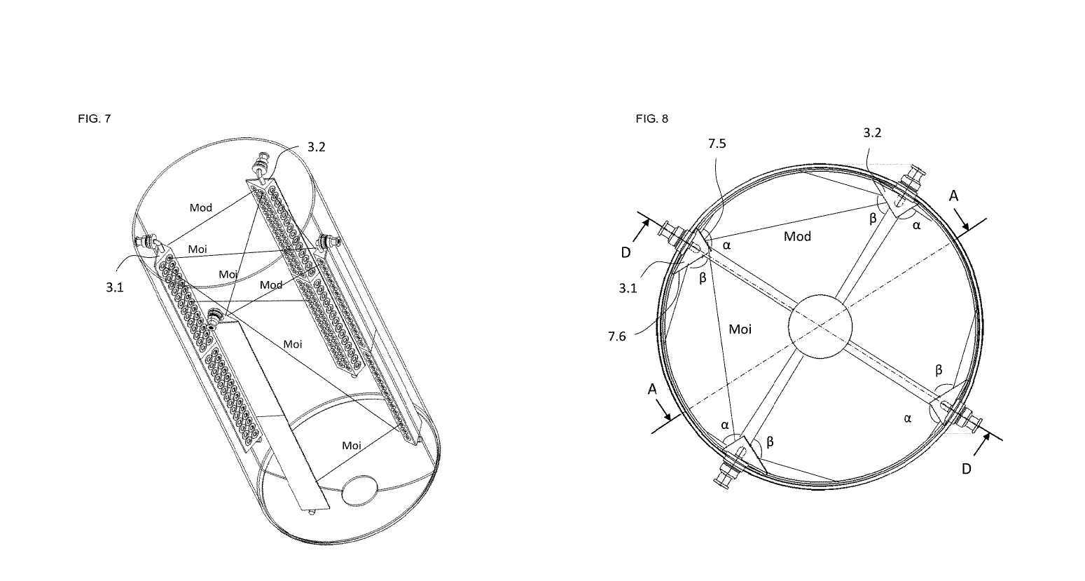

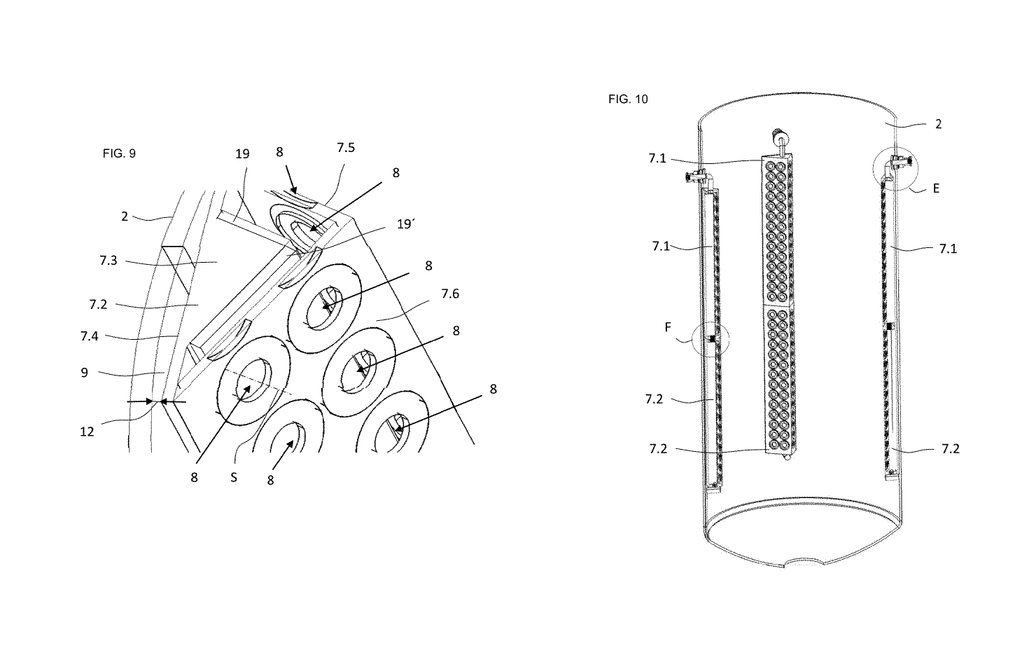

With this choice of angles γ and δ, particularly homogeneous illumination of the container 2 is possible, as will be explained in more detail further below with reference to FIG. 8.

However, this choice of angles γ and δ shall not be considered as limiting, since other angles may be advantageous as well, for example if less than four or more than four devices 3 for emitting electromagnetic radiation are provided in a container 2.

The housing 7, 7.1, 7.2 of the device 3 for emitting electromagnetic radiation preferably accommodates at least one source 13 for emitting electromagnetic radiation and preferably at least one sensor 14 for measuring electromagnetic radiation, which will be described in more detail below with reference to the FIGS. 9, 13, and 14.

FIG. 14 shows an embodiment of a source 13 for emitting electromagnetic radiation comprising a single-row array 14 of LEDs 15 and a single-row array 16 of sensors 17 and a controller 18 for controlling the LEDs 15 and for communication with the sensors 17.

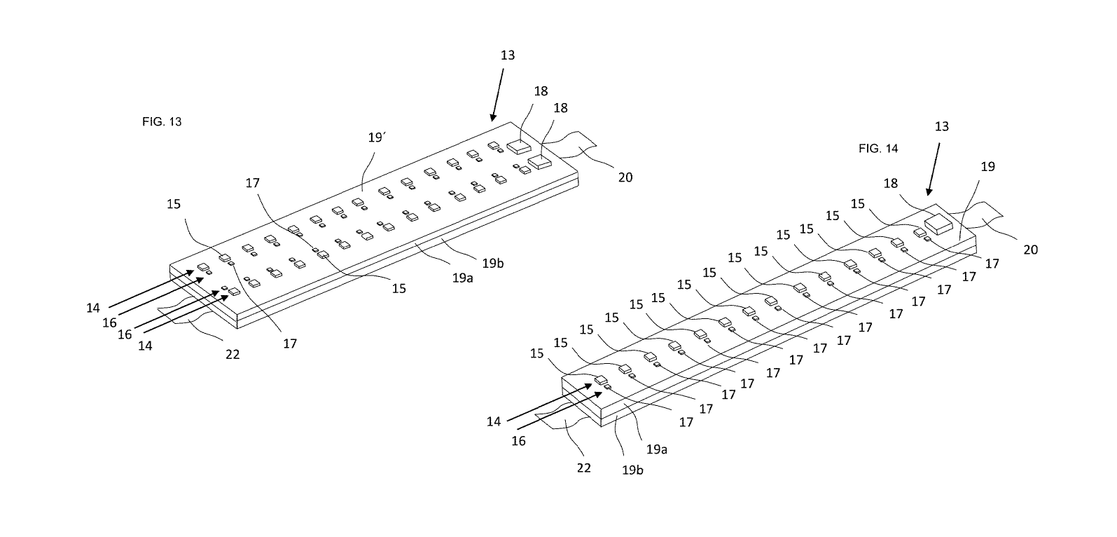

Both the LEDs 15 of the array 14 and the sensors 17 of the array 16 are each conductively connected to the controller 18 through a printed circuit board (PCB) 19 and are mounted to the front of the respective PCB 19, 19′. Since the conductive connections of the PCB 19 extend within the PCB 19, they are not shown in the figures, although known per se to those skilled in the art. In the views of FIGS. 13 and 14, the rear side of circuit boards 19, 19′ respectively faces the paper on which they are illustrated.

FIG. 13 shows a further embodiment of a source 13 for emitting electromagnetic radiation, which in contrast to the embodiment shown in FIG. 14 comprises a two-row array 14 of LEDs 15 and a two-row array 16 of sensors 17 and two controllers 18 for driving the LEDs 15 and communicating with the sensors 17, but which otherwise functions substantially similarly to the embodiment of FIG. 13. Here, this embodiment comprises a printed circuit board (PCB) designated by reference numeral 19′ for distinguishing this PCB from the PCB of FIG. 14 designated by reference numeral 19.

For example, a multi-layer package with printed conductors and ceramic substrates 19a and 19b exhibiting very high thermal resistance and also high electrical conductivity can be provided for the circuit board 19′.

According to a preferred embodiment of the invention, a direct copper bond (DCB) ceramic substrate is used as the ceramic substrate 19a, 19b.

This is a ceramic substrate for which a high-temperature melting and diffusion process is used to apply copper, especially pure copper, on a ceramic insulator to be bonded thereto to firmly adhere to the ceramic. Besides producing a firm bond to the substrate, this allows to apply rather thick conductive tracks, in particular conductive tracks with a thickness of about 200 μm. Therefore, large currents can be carried by the conductive tracks of a DCB substrate.

Another embodiment uses a direct plated copper (DPC) ceramic substrate 19a, 19b. This is a ceramic substrate with a metallic electroplating starter layer, in particular a Cr/Ni layer, onto which a copper layer is applied. This may especially be accomplished using a galvanization process. In this way, layer thicknesses of more than 10 μm, preferably up to 100 μm or more may be produced by galvanization. An embodiment as a direct printed copper ceramic substrate 19a, 19b is also conceivable. In this way, copper layers of more than 10 μm can be applied by a thick layer printing process.

In the operating state, a sensor 17 can be used to measure preferably the intensity and/or wavelength of electromagnetic radiation incident thereon.

The arrangement of the printed circuit board 19 and 19′ can be seen in FIG. 9, for example, with the respective LEDs 15 and sensors 17 facing the windows 8 arranged in front thereof and with the rear side of circuit board 19, 19′ mounted to a housing body 7.3 of the housing 7.2.

Preferably, one LED 15 and one sensor 17 of the respective array 14, 16 are arranged behind a common window 8 in each case.

However, one of the LEDs 15 and/or one of the sensors 17 may also be arranged behind more than one window 8 of the housing 7.1, 7.2.

The controller 18 is connected to further supply and control devices via a multi-conductor link 20 which extends through the interior of the feedthrough and holding device 4 to a connector 21 as shown in FIG. 11, which further supply and control devices are, however, not shown in the figures for the sake of clarity.

For the sake of clarity, only one line of the multi-conductor link 20 is shown connected to the connector 21 in FIG. 11.

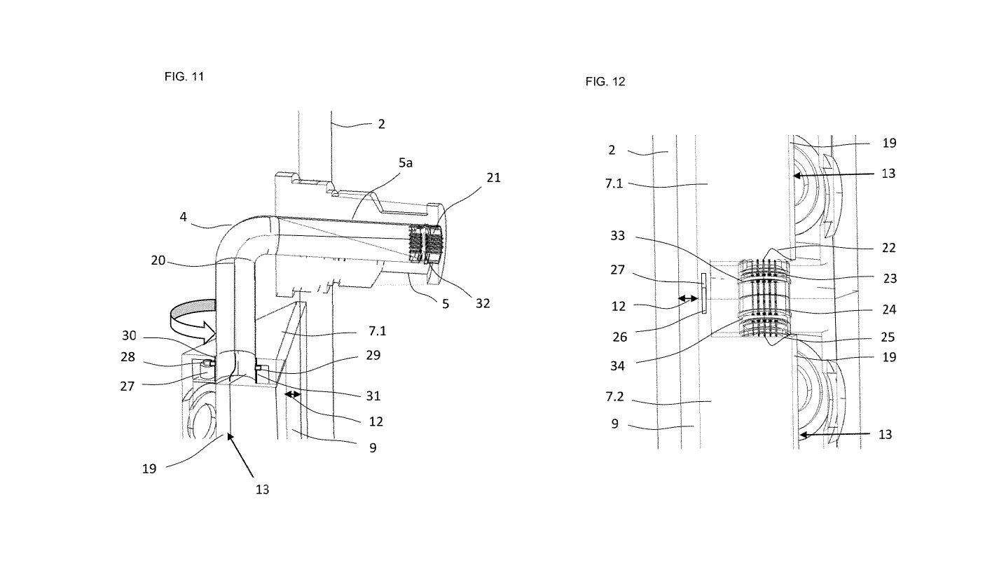

Also for the sake of clarity, only one line of the multi-conductor link 22 is shown connected to the connector 23 in FIG. 12.

The connector 23 of the housing 7.1 of the first module is complementary interengagable with the connector 24 of the housing 7.2 of the second module, so that the housing 7.1 comprises at least one electrical connector which preferably can be serially coupled to the connector 24 of the further housing 7.2.

Via a further multi-conductor link 25, the connector 24 is connected to the printed circuit board 19 of the further source 13 for emitting electromagnetic radiation disposed within housing 7.2, and in particular to the controller 18 arranged on this printed circuit board (but not shown in the figures).

The presently disclosed multi-conductor links each comprise supply lines and/or control lines for the device 3 for emitting electromagnetic radiation and at least one of them extends through at least one feedthrough 5a.

So, both the housing 7.1 and the housing 7.2 are configured to be coupled in modular form, and such modules, by being serially connectable, permit to account for diverse conditions of respective photobioreactors and in particular those already existing.

Housings 7.1 and 7.2 may in particular furthermore comprise fasteners for being coupled in modular form, such as dowel pins of a first housing, which are engageable in associated fitting grooves or tightly fitting blind holes of the respective second housing.

By way of example, FIG. 12 only shows the dowel pin 26 of housing 7.2 of the second module, which engages in an associated bore 27 of housing 7.1 of the first module.

Referring again to FIG. 14, which shows both the multi-conductor link 20 connected to the connector 21 and the multi-conductor link 22 connected to the connector 23. The multi-conductor link 20, 22 as well as the multi-conductor link 25 which connects the connector 24 to the printed circuit board 19 and its controller 18 may comprise both signal lines of a communication bus and respective power supply lines, in particular for the controllers 18 and the LEDs 15 and if necessary also for sensors 17.

A preferred communication bus includes, for example, data exchange via OPC XML and includes JAVA programmable clients. Such a communication bus with its clients is described, for example, in “SIMOTION—Description and example for the data exchange via OPC XML interface”, Version 1.0 Edition 07/2007, published by Siemens AG.

The controller 18 of printed circuit board 19 allows to control the servomotor 27 of the first housing 7.1. The servomotor 27 is stationarily mounted to the housing 7.1 which, however, is mounted on the feedthrough and holding device 4 so as to be rotatable in accordance with the arrow shown in FIG. 11.

Gear 28 driven by servomotor 27 meshes in frictional fit with gear 29 that is firmly connected to the feedthrough and holding device, so by rotating gear 28 under the control of controller 18, the device 3 for emitting electromagnetic radiation can be rotated about its longitudinal axis in the direction of the arrow shown in FIG. 11 in a defined manner. Rotation in the direction opposite to the arrow shown in FIG. 11 is possible by changing the direction of rotation of gear 28.

Such rotation furthermore allows to adjust the measuring distances Mod and Moi that will be described in more detail further below. This adjustment may be controlled manually or by a process control device which communicates with the controllers 18.

In order to ensure hermetic tightness of the housing 7.1, the feedthrough and holding device 4 has a ceramic sealing element 30 and the housing 7.1 has a ceramic sealing element 31.

Each of the connectors 21, 22, and 23 also has at least one ceramic sealing element 32, 33, 34 to thereby ensure hermeticity of the housings 7.1 and 7.2. Moreover, the ceramic sealing elements are thermally stable at temperatures employed during sterilization, in particular at temperatures of 150° C. and more, and are also resistant to chemical cleaning agents such as those conventionally used for the cleaning of photobioreactors or bioreactors.

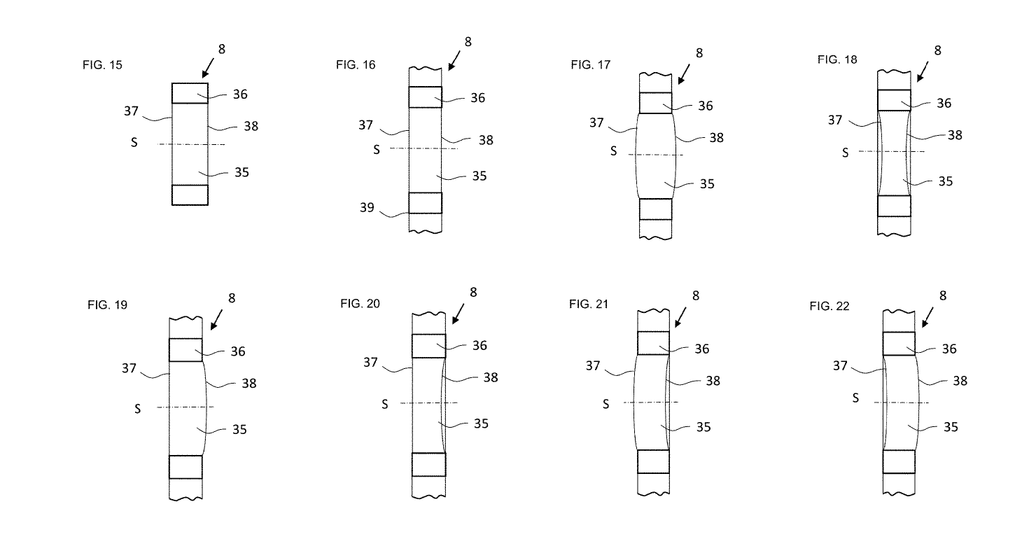

In the present preferred embodiments, a window 8 or a plurality of windows 8 each provide a glass seal for a transparent element 35, preferably a glass-to-metal seal (GTMS) compression glass seal, as will be described below with reference to FIG. 15.

FIG. 15 shows a schematic sectional view along an axis of symmetry S of a window 8 described herein for explaining the method for producing such a window.

The window 8 shown in FIG. 15 comprises an annular or cylindrical main body 36 made of steel, which encloses the transparent element 35 laterally while exerting thereon a compressive force which ensures a permanently hermetic connection between the transparent element 35 and the main body 36 sufficiently pressure-resistant and heat-resistant for the purposes of the present invention.

The transparent element 35 of the respective window 8 comprises glass or is made of glass in each case.

The glass of the transparent element 35 of the windows 8 may comprise or consist of a quartz glass or else of a borosilicate glass, for example.

For producing such a window, the transparent element 35 is arranged within the main body 36, preferably in approximately its final shape, and is heated together with the main body until the glass of the transparent element 35 has exceeded its glass transition temperature T_{g} or hemisphere temperature and begins to fuse to the main body 36.

Once fused, the assembly of transparent element 35 and main body 36 is then cooled to room temperature, thereby forming a respective window 8 that includes a substantially sheet-like transparent element 35.

This process, described above, in which the glass of the transparent element 35 is connected to the main body 36, is known to the person skilled in the art to be referred to as glassing, and the resulting connection of the transparent element 35 to the main body 36 is referred to as Einglasung.

Since the stainless steel of the main body 36 has a thermal expansion coefficient that is greater than that of the glass of the transparent element 35, it will exert a compressive stress to the glass of the transparent element 35 as soon as the glass of the transparent element begins to solidify, which compressive stress is increasing with decreasing temperature.

Once the cooling has been completed, the main body 36 will then permanently and reliably hold the transparent element 35 in a hermetically sealed and temperature-stable manner due to this quasi-frozen compressive stress.

Such a compression glass seal is also referred to as a Glass-To-Metal Seal, GTMS, in the present disclosure if the main body 36 is made of metal.

In such a glass-to-metal joint, the metal exerts pressure forces on the glass over the entire range of operating temperatures, in particular even at temperatures up to at least 120° C., preferably even up to 140° C., which pressure forces cause a compressive stress between the metal and the glass and help to ensure that the glass-to-metal joint remains permanently and reliably fluid-tight as well as hermetically sealed.

Furthermore, no gaps will arise with such glass-to-metal joints. By contrast, if conventional sealing means such as O-rings are used, gaps may arise and may provide room for contamination that is often difficult to remove.

For this purpose, a difference in the coefficients of thermal expansion is advantageous, which reliably maintains the compressive stress between the glass of the transparent element 35 and the metal of the annular or cylindrical main body 36 of the window 8 at least over the range of operating temperatures.

This difference between the expansion coefficient CTE_{M} of the metal and the expansion coefficient CTE_{G} of the glass of the transparent element 35 may be less than 80×10⁻⁶/K, for example, preferably less than 30*10⁻⁶/K, or most preferably less than 20*10⁻⁶/K. Here, the coefficient of thermal expansion of the metal CTE_{M} should be greater than the coefficient of thermal expansion of the glass CTE_{G} in each case. In any cases, however, this difference should preferably be at least 1*10⁻⁶/K.

For example, quartz glass has a CTE_{G} of 0.6* 10⁻⁶/K and can be combined, for example, with stainless steels having a CTE_{M} of 17 to 18*10⁻⁶/K.

If the housing body 7.3 comprises or is made of a high temperature resistant plastic, in particular a thermoplastic material such as polyaryletherketone, in particular polyetheretherketone, PEEK, this housing body 7.3 need not necessarily have to be completely hermetic as described in the context of the present disclosure, but nevertheless it will be possible to achieve quite valuable operating and application durations.

For example, in terms of a ratio of diameters, the housing body 7.3 may have a preferably circular through-opening of a diameter that is smaller by 1/10 than the outer diameter of the transparent element 35. When the housing body 7.3 is heated to a temperature of about 200° C., the transparent element 35 can then be inserted into this through-opening, and when being cooled down, a compressive stress is resulting as described above, for example of about 38 MPa, which is still well below the yield strength of PEEK of 110 MPa.

In this durable hermetically sealed and temperature-stable state appropriate for continuous operation, the glass of the transparent element 35 can either be used directly, preferably after verifying the respective side face, or may be subjected to further surface processing procedures such as polishing or shaping grinding.

In this way, the respective transparent element 35 may have plane-parallel main side faces 37 and 38, or else the transparent element 35 may be shaped to become one of plano-convex, plano-concave, biconvex, biconcave, convexo-concave, or concavo-convex, as can be seen in the sectional views of FIGS. 16 to 22 in which it is assumed that the LEDs 15 are located to the left of the transparent element 35 in each case.

If there are lower optical requirements imposed on the surface quality, in particular for illuminating beam paths or else for beam paths used for measurement, the transparent element 35 may also be held in a corresponding negative mold which substantially already corresponds to the final shape thereof, during the fabrication process.

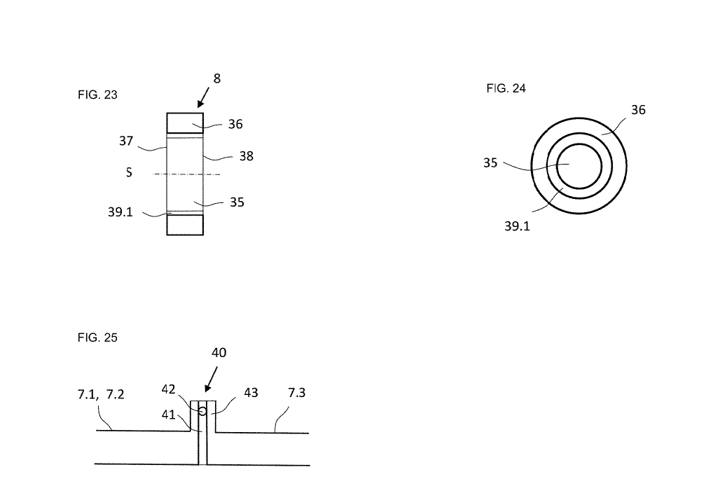

If the transparent element 35 of the window 8 consists of quartz glass or is made of quartz glass, as shown in FIGS. 23 and 24, for example, it is also possible to use a further glass 39.1 or glass solder 39.1 to hermetically seal the quartz glass to the annular or cylindrical main body 36 of the window 8 in a fluid-tight and hermetical manner.

The glass solder used in this case is preferably a glass solder that is free of heavy metals, in particular a lead-free glass solder.

The photobioreactor 1 and the device 3 for emitting electromagnetic radiation are each autoclavable individually, or else the photobioreactor 1 and the device 3 for emitting electromagnetic radiation are also autoclavable together. This means that in particular the device 3 for emitting electromagnetic radiation including its windows 8 as disclosed herein is hermetically sealed so as to withstand a treatment with saturated steam at a temperature of 121° C. without a risk of ingress of saturated steam or fluids generated thereby into the device 3.

In the context of the present disclosure, autoclavable is understood to mean autoclavable in the sense of DIN EN ISO 14937; EN ISO 17665, which applies to medical devices.

This also allows for the advantageous steaming-in-place (SIP) which is known to those skilled in the art.

It is also advantageous if a reduced pressure is prevailing in the housing 7, in particular in housings 7.1 and 7.2 of the modules of the device 3 for emitting electromagnetic radiation, relative to the exterior of the housing 7 of the device 3 for emitting electromagnetic radiation.

This not only reduces heat conduction within the housing 7, 7.1, 7.2, but also reduces corrosion of the assemblies arranged within the housing 7, 7.1, 7.2.

FIG. 25 shows a cross-sectional view through part of the housing 7.1 of the upper or first module of the device 3 for emitting electromagnetic radiation or else of the housing 7.2 of the lower or second module of the device 3 for emitting electromagnetic radiation, which may both have an evacuation port 40.

The evacuation port 40 comprises a through-opening 41 through a wall of the housing 7.1 of the upper or first module of the device 3 for emitting electromagnetic radiation or else of the housing 7.2 of the lower or second module of the device 3 for emitting electromagnetic radiation.

In the view of FIG. 25, evacuation port 40 has a stainless steel ball 42 pressed into the through-opening 41 thereby closing it in a fluid-tight and hermetically sealed manner.

This stainless steel ball 42 may for example be pressed into the through-opening 41 once a reduced pressure has been generated in the housing 7.1, 7.2 of the device 3 for emitting electromagnetic radiation compared to the exterior of the housing, for example after evacuation of the housing.

As an alternative to the use of stainless steel ball 42, a tubular protruding portion 43 of the evacuation port 40 may be sealed by crimping, soldering or welding after the evacuation, in order to provide a fluid-tight and hermetic seal.

The main surfaces 37, 38 of the transparent element 35 of the windows 8 may have a wavelength-selective coating which thereby provides an optical bandpass or edge filter. With such an optical filter, predefined wavelengths may be irradiated into the container 2 by the device 3, and identical wavelengths or different wavelengths may be measured, in particular captured by a sensor. This coating may be provided only on one or else on both main surfaces.

Without such a coating, in particular without any coating, the transparent element 35 of at least one window 8 exhibits a transmittance of greater than 80%, most preferably greater than 90%, in a spectral range of wavelengths between 250 and 2000 nm.

Preferably, the main body 36 of the windows 8 is directly bonded to the housing body 7.3 of the respective housing 7, 7.1, 7.2 of the device 3 for emitting electromagnetic radiation, in particular by laser welding, so that the housing 7, 7.1, 7.2 is hermetically sealed thereby.

In the sense of the present disclosure, an item such as the device 3 for emitting electromagnetic radiation shall be considered as hermetically sealed or fluid-tight if it exhibits a leak rate of less than 1*10⁻³ mbar·1/sec at room temperature when filled with He and exposed to a pressure difference of 1 bar.

The laser welding seam formed thereby is designated by reference numeral 39 in FIG. 16, by way of example.

If, now, light is emitted by one of the LEDs 15, it will pass through the window 8 arranged in front of this LED 15, and the light distribution shown in FIG. 8 will be resulting.

A respective light cone with an opening angle a will be formed in front of a longitudinally extending side wall 7.5 of the housing 7, 7.1, 7.2, which side wall comprises a line of windows 8 and has the source 13 for emitting electromagnetic radiation as shown in FIG. 14 disposed behind these windows.

Furthermore, a respective light cone with an opening angle β will be formed in front of a longitudinally extending side wall 7.6 of the housing 7, 7.1, 7.2, which side wall comprises two lines of windows 8 and has the source 13 for emitting electromagnetic radiation as shown in FIG. 13 disposed behind these windows 8.

These light cones with opening angles α and β include more than 95% of the radiation intensity of the electromagnetic radiation emitted by the respective LED 15, and their spatial shape is substantially defined by the LED 15 and by the transparent optical element 35 arranged in front thereof.

As can be seen in FIG. 8, almost the entire interior of the container 2 is illuminated in this way.

By appropriately shaping the main surfaces 37 and 38 and in combination with the emission characteristic of the respective LED it is possible to achieve a very homogeneous irradiation in which the intensity of the emitted electromagnetic radiation within the light cones with opening angles α and β does not fall below a predefined minimum value of about 30% of the maximum intensity when the container 2 is empty and losses attributable to scattering and absorption and the influence of reflection are negligible.

Furthermore, the LEDs 15 of a first device 3.1 for emitting electromagnetic radiation, which is only shown in FIGS. 7 and 8 by way of example, together with the sensors 17 of a second device 3.2 for emitting electromagnetic radiation, define a predefined measuring device, for instance for capturing scattering and absorption losses that occurred along the measuring distance Mod defined by the two devices 3.1 and 3.2. This allows to perform in situ measurements corresponding to a turbidity chamber.

Furthermore, this measurement is not limited to only two devices for emitting electromagnetic radiation, rather, as shown in FIG. 7 by way of example, such measurements may also be performed between a plurality of other LEDs 15 and sensors 17, as indicated by further measuring distances Mod and Moi in FIG. 7, for example.

In addition to the measuring distance with a direct optical link, as indicated by the measuring distance Mod in FIG. 8 and for which the respective measuring sensor 17 for this measuring distance Mod is located within the light cone with opening angle α or β so that it would be directly illuminated by the LED 15 associated with this measuring distance Mod if there was no fluid 9 in the container 2, there are also measuring distances Moi with practically indirect illumination of the optical sensor 17 associated with this measuring distance, which are again only shown in FIG. 7 by way of example.

For these measuring distances Moi, the sensor 17 associated with this measuring distance is located outside the light cone of opening angle α or β of the light-emitting diode 15 associated with this measuring distance, but can nevertheless capture scattered light which is produced by the fluid 9 including the biological material along this respective measuring distance.

Since the electromagnetic radiation emitted by the LEDs 15 may also be emitted in short pulses, the subsequent measuring cycle may be carried out over a predefined time period.

One or a predefined number of first light-emitting diodes 15 emit electromagnetic radiation of a defined intensity for a short time interval, for example from 1 to 10 μs. During this time interval, a sensor 17 or a plurality of sensors 17 can measure the received intensity of electromagnetic radiation.

Thereafter, a second light emitting diode may emit electromagnetic radiation of a defined intensity for a short time interval, for example from 1 to 10 μs. Again, a sensor 17 or a plurality of sensors 17 can measure the received intensity of the electromagnetic radiation during this time interval.

This procedure can be continued until the interior of the container 2 has been completely captured with the desired spatial and time resolution, in particular also in its dynamic or static behavior.

This allows for sensory capturing optimized in time response and based thereon for excellent responsiveness in the optimization of the processes within the container 2 of the photobioreactor 1 and also provides for spatially resolved sensory capturing, and in this way an optimization can come to fruition throughout the entire captured volume within the container 2 of the photobioreactor, in particular a controlled optimization of the processes within the container 2 of the photobioreactor 1.

Appropriate driving of the LEDs 15 and evaluation of the sensory parameters captured by the sensors 17 is effected using the controllers 18 and can be communicated to and controlled by process control devices and associated evaluation devices external to the container 2, via the multi-conductor links.

In a further preferred embodiment, an N·M array of LEDs 15 and sensors 17 is used in combination with respective windows 8 arranged in front thereof, wherein each of N and M can preferably range from 2 to 125.

Furthermore, when emitting a plane wave, for example if the LED 15 is a laser diode and a plane-parallel transparent element is used having an opening angle of almost 0°, a second associated sensor array can be used to perform a line by line measurement inside the container 2 which may capture almost the entire internal volume of the container 2.

This allows for a particularly advantageous regulation of the real actual intensity of the electromagnetic radiation in the fluid. Furthermore, dynamic adaptation to a target intensity is possible. A compensation of the varying illumination depth that might be caused by the changing/increasing cell density during the cultivation is made possible in real time or at least with only a slight time offset.

Also, a compensation for substrate consumption and/or for adsorption changes in the fluid due to product formation is possible with both spatial and temporal resolution.

So, with the devices 3, 3.1 and 3.2 described herein, the photobioreactor 1 allows for a temporally and spatially resolved in situ measurement of optical absorption, scattering and fluorescent signals from the biological material 10 and for use of such measurement signals to optimize the desired metabolic processes of the biological material 10.

Based on these measurement signals, the supply of nutrient solution may be effected more efficiently with both spatial and temporal resolution, if the photobioreactor comprises for instance appropriate further supply devices not shown in the figures.

Furthermore, an in situ efficiency measurement may be performed along each of the measuring distances described above, in the form of a wavelength penetration depth measurement as a growth indicator of the biological material 10.

The photobioreactor 1 may additionally comprise agitators for moving the fluid medium in a manner known to those skilled in the art, though not shown in the figures, for the sake of clarity. Furthermore, the photobioreactor may comprise feeders, tempering means and flow-influencing means, not shown either in the figures, although known to those skilled in the art.

The modules of the device for emitting electromagnetic radiation described herein are preferably mounted in the most favorable area of flow distribution in the bioreactor, in particular in the case of photobioreactors with agitators. Compared to tube reactors, a more homogeneous distribution of the light quanta on the cells is resulting so in the statistical mean.

The devices described herein in particular provide for an upscale and transfer from laboratory scale to production scale of cultivation of phototrophic microorganisms under sterile conditions.

However, the invention encompasses not only devices but also methods.

One of these methods is a method for propagation or cultivation of biological material, preferably for producing pharmaceuticals, in particular biopharmaceuticals, which comprises: the introducing of biological material or of a precursor of biological material into a photobioreactor as disclosed herein, the exposing to electromagnetic radiation emitted by a device for emitting electromagnetic radiation as disclosed herein.

Another method comprises a method for preparing biological material and/or for producing pharmaceuticals, in particular biopharmaceuticals, which comprises: providing a photobioreactor 1 and equipping it with at least one device for emitting electromagnetic radiation as disclosed herein.

In these methods, the photobioreactor 1 has at least one feedthrough 5a, in particular at least one standard sparger port 5, and supply lines and/or control lines are routed through the at least one feedthrough 5a, in particular a multi-conductor link 20 for the device 3 for emitting electromagnetic radiation. In this way, a controller 18 that is preferably arranged on the printed circuit board 19, 19′ of a source 13 for emitting electromagnetic radiation can communicate with or be controlled by process control devices and associated evaluation devices external to the container 2, for example.

The printed circuit board 19 may consist of a material that exhibits a temperature resistance of 380° C. here, for example, which is particularly advantageous for cycle resistance during autoclaving.

In the methods disclosed herein, the photobioreactor 1 comprises at least one mounting means 6 for fixing baffles or turbulence metal sheets in its interior, in particular in the interior of the container 2 for holding biological material, and the at least one device for emitting electromagnetic radiation is connected to at least this one mounting means 6, and the device 3 for emitting electromagnetic radiation is preferably fixed in the interior of the photobioreactor 1, in particular in the interior of the container 2 for holding biological material through the at least one feedthrough and the at least one mounting means.

Generally and in particular when carrying out the methods disclosed herein, the photobioreactor 1 can be sterilized even after having been equipped with the device 3 for emitting electromagnetic radiation.

In the present methods, at least one device 3 for emitting electromagnetic radiation advantageously comprises at least one sensor 17 for measuring electromagnetic radiation, and in the operating state the radiation intensity and/or wavelength of the electromagnetic radiation is measured inside the container 2 of the photobioreactor 1, and the radiation intensity and/or wavelength of the electromagnetic radiation inside the container 2 of the photobioreactor 1 is preferably measured in a time resolved and spatially resolved manner during the operating state.

Furthermore, in the methods described herein, electromagnetic radiation of a defined wavelength, preferably 250 nm, may be irradiated into the photobioreactor for a predefined period of time, and after this irradiation, a radiation intensity and/or wavelength of the electromagnetic radiation inside the container 2 of the photobioreactor 1 can be measured over a broad range of wavelengths.

Furthermore, electromagnetic radiation of a defined wavelength, preferably 250 nm, may be irradiated into the photobioreactor for a predefined period of time, preferably during a production phase, and after this irradiation, electromagnetic radiation of a defined further wavelength may be irradiated, preferably in a range from 620 to 780 nm.

Advantageously, photo- or mixotrophic microorganisms modified by mutagenesis can in particular be used in these methods, inter alia.

The methods described herein may be performed in a batch mode, in a fed-batch mode with defined feed rate, in particular with a spatially and temporally resolved and controlled feed rate, and by irradiation of suitable wavelengths, for example of 250 nm, inhibition, in particular substrate inhibition may advantageously be caused, which results in an increased production rate of the biological material.

A stimulation of product formation may also be achieved by temporally and spatially resolved and preferably controlled supply of nutrient solution, in particular sugar supply and refeeding.

The methods described herein may also be employed in a repeated batch mode.

Since the optima of wavelengths are always strain-specific, this can be accounted for by an appropriate selection of the LEDs 15 or laser diodes 15 and by the coating of the transparent element 35 described above.

Besides production, the invention is also very advantageous for process development. Especially for the latter, a flexible adaptation of the wavelengths can bring further advantages.

In order to provide a photobioreactor with a device for emitting electromagnetic radiation as well as a device for emitting electromagnetic radiation for a photobioreactor which allow the most efficient use of the photobioreactor and contribute to reduce contamination of the interior of the photobioreactor, it is generally suggested according to the invention that the device for emitting electromagnetic radiation including its housing is arranged inside the container for holding biological material.

Advantageously, the device for emitting electromagnetic radiation may also have one or more sensors for measuring the CO2 content within the bioreactor.

The present application also encompasses the subject matter of the applications of the present Applicant entitled “Sensor receptacle for a bioreactor and bioreactor with sensor receptacle and method for propagation or cultivation of biological material” and entitled “Device for supporting an image capturing device on a bioreactor, bioreactor with device for supporting an image capturing device, and method for propagation or cultivation of biological material”, which were filed with the German Patent and Trademark Office on the same day as the present application and which are fully incorporated into the present application by reference.

LIST OF REFERENCE NUMERALS

| | |
|---|---|
| 1 | Photobioreactor |
| 2 | Container |
| 3 | Device for emitting electromagnetic |
| | radiation |
| 4 | Feedthrough and holding device |
| 5 | Sparger port |
| 6 | Mounting means |
| 7 | Housing |
| 7.1 | Housing of the upper or first module |
| 7.2 | Housing of the lower or second module |
| 7.3 | Housing body |
| 7.4 | Side wall |
| 7.5 | Side wall |
| 7.6 | Side wall |
| 8 | Window |
| 9 | Fluid medium |
| 10 | Biological material |
| 11 | Surface of fluid medium |
| 12 | Minimum distance |
| 13 | Source for emitting electromagnetic |
| | radiation |
| 14 | Single-row array of LEDs 15 |
| 15 | LEDs |
| 16 | Single-row array of sensors 17 |
| 17 | Sensor |
| 18 | Controller |
| 19, 19′ | Printed circuit board (PCB) |
| 19a | Ceramic substrate |
| 19b | Ceramic substrate |
| 20 | Multi-conductor link |
| 21 | Connector |
| 22 | Multi-conductor link |
| 23 | Connector |
| 24 | Connector |
| 25 | Multi-conductor link |
| 26 | Dowel pin |
| 27 | Servomotor |
| 28 | Drive gear |
| 29 | Output gear |
| 30 | Ceramic sealing element |
| 31 | Ceramic sealing element |
| 32 | Ceramic sealing element |
| 33 | Ceramic sealing element |
| 34 | Ceramic sealing element |
| 35 | Transparent element |
| 36 | Base body |
| 37 | Main side face |
| 38 | Main side face |
| 39 | Laser welding seam |
| 39.1 | Glass solder |
| 40 | Evacuation port |
| 41 | Through-opening |
| 42 | Stainless steel ball |
| 43 | Protruding tubular portion |

What is claimed is:

1. A photobioreactor, comprising:
a container for holding fluid media containing biological material; and
an electromagnetic radiation emitting device comprising a housing with a source for emitting electromagnetic radiation arranged therein,
wherein the electromagnetic radiation emitting device including the housing thereof is arranged inside the container for holding biological material.

2. The photobioreactor of claim 1, comprising a feedthrough extending through at least one wall of the photobioreactor, wherein the electromagnetic radiation emitting device is held on the feedthrough.

3. The photobioreactor of claim 1, wherein the electromagnetic radiation emitting device comprises a plurality of electromagnetic radiation emitting devices, the plurality of electromagnetic radiation emitting devices being arranged inside the container symmetrically relative to a longitudinal axis of the container.

4. The photobioreactor of claim 1, wherein the container comprises stainless steel or is made of stainless steel.

5. The photobioreactor of claim 1, wherein the electromagnetic radiation emitting device including the housing thereof is arranged spaced apart from a wall of the container.

6. The photobioreactor of claim 1, further comprising a mount in an interior of the container, the mount being positioned and configured to fix baffles and/or turbulence metal sheets, wherein the electromagnetic radiation emitting device is connected to the mount.

7. The photobioreactor of claim 1, wherein the container and the electromagnetic radiation emitting device including the housing thereof are autoclavable together.

8. A device for emitting electromagnetic radiation, comprising:
a housing; and
a source for emitting electromagnetic radiation arranged therein, wherein the source comprises an LED.

9. The device of claim 8, further comprising a sensor arranged in the housing, the senor being configured to measure electromagnetic radiation.

10. The device of claim 9, wherein the senor is configured to adjust an intensity and/or wavelength of incident electromagnetic radiation.

11. The device of claim 10, wherein the housing further comprises a window, the LED and/or the sensor being arranged behind the window.

12. The device of claim 11, further comprising a glass-to-metal seal (GTMS) compression glass seal sealing the window and the housing.

13. The device of claim 11, wherein the window is directly connected to the housing in a hermetically sealed manner by a laser weld.

14. The device of claim 11, wherein the window has a transparent element, the transparent element having a shape selected from a group consisting of a sheet-like shape with plane-parallel main surfaces, plano-convex, plano-concave, biconvex, biconcave, convexo-concave, and concavo-convex.

15. The device of claim 14, wherein the transparent element comprises a material selected from a group consisting of glass, quartz glass, and borosilicate glass.

16. The device of claim 14, wherein the transparent element exhibits a transmittance of greater than 80% in a spectral range of wavelengths between 250 and 2000 nm.

17. The device of claim 8, wherein the housing has a columnar shape with longitudinally extending side walls, wherein two of the longitudinally extending side walls enclose an angle of 30° and two of the longitudinally extending side walls thereof enclose an angle of 60°.

18. The device of claim 17, wherein at least two of the longitudinally extending side walls have windows.

19. The device of claim 8, wherein the housing further comprises an electrical connector.

20. The device of claim 8, wherein the housing further comprises an evacuation port that can be sealed fluid-tightly, wherein the housing has pressure therein that is reduced relative to the pressure exterior of the housing.

21. A method for propagation or cultivation of a biological material, a pharmaceutical, or a biopharmaceutical, comprising:
introducing the biological material, pharmaceutical, or biopharmaceutical or a precursor thereto into a photobioreactor, the photobioreactor having an electromagnetic radiation emitting device comprising a housing with a source for emitting electromagnetic radiation arranged in the housing, the housing being arranged in an interior of the photobioreactor; and
emitting electromagnetic radiation from the electromagnetic radiation emitting device into the photobioreactor.

22. The method of claim 21, further comprising routing supply lines and/or control lines for the electromagnetic radiation emitting device through a feedthrough of the photobioreactor.

23. The method of claim 21, further comprising mounting the electromagnetic radiation emitting device to a baffle and/or turbulence metal sheets in the interior of the photobioreactor.

24. The method of claim 21, further comprising sterilizing the photobioreactor having the electromagnetic radiation emitting device including the housing arranged in the interior.

25. The method of claim 21, further comprising measuring a radiation intensity and/or a wavelength of electromagnetic radiation inside the photobioreactor during operation.

26. The method of claim 21, further comprising:
irradiating electromagnetic radiation of a predefined wavelength into the photobioreactor for a predefined period of time; and
measuring, after irradiating, a radiation intensity and/or a wavelength of the electromagnetic radiation inside the photobioreactor over a broad range of wavelengths or selectively at a particular wavelength.
